(19) United States
(12) Patent Application Publication
(10) Pub. No.: US 2007/0276484 A1
(43) Pub. Date: Nov. 29, 2007

(54) METHOD AND APPARATUS FOR INSERTION OF A FLEXIBLE IMPLANT INTO THE HUMAN BODY

(75) Inventors: Edward Alan Abell, Gainesville, GA (US); Nabil L. Muhanna, Gainesville, GA (US); David L. Schalliol, Oakwood, GA (US)

Correspondence Address: ADAMS EVANS P.A., Suite 2350 Charlotte Plaza, 201 South College Street, CHARLOTTE, NC, 28244, US

(73) Assignee: INSERTION, LLC, GAINESVILLE, GA (US)

(21) Appl. No.: 11/672,627

(22) Filed: Feb. 8, 2007

Related U.S. Application Data
(60) Provisional application No. 60/803,270, filed on May 26, 2006.

(51) Int. Cl. A61F 2/12

(52) U.S. Cl. 623/7

(57) ABSTRACT

An insertion device for inserting an implant into a body through an incision is provided. The device includes a holder for receiving an implant and has an open downstream end that is dimensioned to be inserted into the incision and for passing the implant therethrough. The device also includes a flexible carrier that extends through the downstream end of the implant holder and is positioned along inner and outer wall surfaces of the holder. The carrier is dimensioned to frictionally engage the implant at least at a position on the inner wall surface proximate the downstream end of the holder. The carrier and the holder are configured such that when the carrier is caused to move through the open downstream end, the implant moves with the carrier, through the opening, and into the incision.

### TECHNICAL FIELD AND BACKGROUND OF THE INVENTION

This invention relates to a method and apparatus for inserting a flexible implant into a human or animal body. Insertion is accomplished through a relatively small incision in the body and utilizes a fabric carrier and funnel-shaped introducer with a retrograde propulsion system. For purposes of illustration, the invention is described with reference to a breast implant. However, the invention may have application in the insertion of other types of implants, and these other uses are within the scope of the invention.

Silicone breast implants were the primary means of breast augmentation in the U.S. and worldwide until 1992, when the FDA removed them from the market for use in primary breast augmentation. During that time silicone breast implants became one of the most studied types of medical devices in history. In 2005, the FDA decided that the initial concerns about silicone breast implants causing auto-immune diseases were unfounded or overstated and are in the process of releasing silicone breast implants for use in breast augmentation again.

Since the removal of silicone breast implants from the market by the FDA, they have been replaced by saline-filled implants. Saline implants have several disadvantages relative to silicone implants. Namely, saline implants are heavier, less natural in appearance, less natural feeling, and can cause a more rippled appearance than silicone implants.

There are also advantages in using saline implants. If a saline implant ruptures, there is no concern about the contents leaking out into the body unlike silicone implants. Deflated saline implants can easily be placed through a small incision and inflated once in the patent, allowing the implant to be placed through a smaller incision with far less trauma to the implant. The saline implants also have a significantly decreased incidence of capsular contracture or scarring around the implant, which can make the implants appear hard and occasionally cause pain and deformity.

One of the major causes of capsular contracture is thought to be a mild subacute infection or colonization of the implant with bacteria that are killed by the body's immune system. A consequence of this immune reaction is inflammation and thus formation of a thick firm scar around the implant. It is believed that the primary source of the bacteria is from the patients' skin, in spite of standard preoperative antiseptic preparation of the skin. The bacteria lives just below the outermost layer of skin and is therefore protected from the antibacterial solution used to clean the skin preoperatively. When the skin is rubbed the bacteria is freed. There are numerous other sources of bacteria such as gloves and instruments. Other substance such as powder on gloves can also cause similar inflammation and increased scarring.

Though the initial concern about silicone breast implants causing auto-immune disease seems to have been settled, there are still concerns about their use. One of the factors that have allowed silicone implants to be used again is the fact that the newer implants are made of a cohesive gel silicone. The silicone in the new implants is ideally a very soft solid, like gelatin, so that if the shell ruptures the silicone is less likely to leak out into the body. Therefore the implants must be filled at the time of manufacture, unlike saline implants which are deflated and then filled inside the body. Saline implants can commonly be placed through a 3 cm incision or smaller. In contrast, a silicone implant can require incisions up to 6 cm.

When the silicone implant is forced through the more desirable small incisions, it is done with considerably more pressure and trauma than is required with a saline implant. The added trauma may result in weakening or tearing of the implant shell. The amount of pressure used is dependent upon the surgeon, the size of the implant and the size of the incision. There is no way to consistently control or monitor the amount of pressure or trauma used in each case.

When forcing an implant through the incision in the skin, it often rubs against the skin with variable amounts of force which results in exposure to the bacteria on the skin. Ideally there is as little contact with the implant as possible in order to limit exposure to anything that may cause inflammation around the implant that would decrease the risk of significant capsular contracture. However, this can only be achieved to a limited extent with silicone implants.

Placing the silicone implants can be quite difficult and time consuming in inexperienced hands. Due to the fact that silicone implants have had limited use for breast augmentation in recent years, the majority of surgeons who have been trained in the past twelve to fifteen years have little or no experience with silicone implants. These same surgeons will soon face patients who desire the superior appearance and feel of silicone implants, but desire the smaller incision which has become commonplace as a result of the use of saline-filled implants. This procedure should be achieved in a safe, effective manner, should be easily controlled and should limit risk to the patient and liability to the surgeon.

### SUMMARY OF THE INVENTION

Therefore, it is an object of the invention to provide a method and apparatus for inserting a flexible implant into a human or animal body.

It is another object of the invention to provide a method and apparatus for inserting a flexible breast implant into a human body.

It is another object of the invention to provide a method and apparatus for inserting an implant into a human or animal body that reduces the likelihood of infection proximate the insertion side.

It is another object of the invention to provide a method and apparatus for inserting an implant into a human or animal body that permits insertion through a smaller insertion than would otherwise be required.

It is another object of the invention to provide a method and apparatus for inserting an implant into a human or animal body that provides more uniform, predictable and desirable results than with conventional techniques.

These and other objects and advantages of the invention are achieved in an embodiment of the invention that, for illustrative purposes, includes a solid funnel with a rotating propulsion device. The implant to be introduced is placed in a tubular, flexible fabric carrier. The implant and carrier are placed in the funnel. Straps on the flexible tubular carrier are passed through the small end of the funnel and wrapped around the outside of the funnel to the rotating propulsion device. The propulsion device is secured to the back of the funnel and is configured to produce traction on the carrier. The small end of the funnel is placed into the wound. The propulsion device pulls the straps and carrier out of the smaller opening in the distal end of the funnel shape device and back along the outside of the device. This pulls the carrier and implant through the opening in the device, allowing the implant to be propelled forward through the incision in the skin while pulling the carrier out of the wound and back toward the propulsion device.

Surgery using the device is performed in a manner similar to surgery without the device. The pocket in which the implant is to be placed is created just as it would be normally. When the implant is to be placed in the pocket, the use of the device greatly reduces implant trauma and contamination relative to standard techniques. The process can also be used for saline filled implants with a smaller and narrow funnel.

These and other objects and advantages of the invention are disclosed below, where an insertion device for inserting an implant into a body through an incision is provided. The device includes a holder for receiving an implant and has an open downstream end that is dimensioned to be inserted into the incision and for passing the implant therethrough. The device also includes a flexible carrier that extends through the downstream end of the implant holder and is positioned along inner and outer wall surfaces of the holder. The carrier is dimensioned to frictionally engage the implant at least at a position on the inner wall surface proximate the downstream end of the holder. The carrier and the holder are configured such that when the carrier is caused to move through the open downstream end, the implant moves with the carrier, through the opening, and into the incision.

According to one embodiment of the invention, the device also includes a propulsion device that cooperates with the flexible carrier for propelling the implant from within the implant holder through the downstream end and into the incision by movement of the flexible carrier. A portion of the flexible carrier moves outside of the holder away from the downstream end and, simultaneously, another portion of the flexible carrier engages the implant and moves inside the holder toward the downstream end. The movement of the flexible carrier causes the implant to move toward the downstream end.

In another embodiment of the invention, the propulsion device includes a winding cylinder that is attached to the holder.

In another embodiment of the invention, the carrier extends through the downstream end of the holder and is attached to the winding cylinder.

In another embodiment of the invention, a crank is attached to the winding cylinder for rotating the winding cylinder, thereby pulling the carrier through the end of the holder.

In another embodiment of the invention, the carrier includes a flexible sleeve that is dimensioned to receive the implant therein.

In another embodiment of the invention, the holder is funnel shaped and has an open upstream end that is larger than the downstream end.

In another embodiment of the invention, an insertion device for inserting an implant into a body through an incision is provided that includes a tubular implant holder for retaining an implant therein. The tubular implant holder has a downstream, open end that is dimensioned to be inserted into the incision and the implant passed in a downstream direction through the open end from a position upstream therefrom. The insertion device also includes a flexible carrier that extends through the open end of the implant holder and that is positioned along inner and outer wall surfaces of the implant holder for frictionally-engaging the implant at least at a position on the inner wall surface proximate the open end of the implant holder. A propulsion device is configured to cooperate with the flexible carrier. The flexible carrier is adapted for propelling the implant from within the implant holder through the downstream open end and into the incision by upstream movement of the flexible carrier relative to the outer wall surface of the implant holder and simultaneous corresponding downstream implant-engaging movement of the flexible carrier relative to the inner wall surface of the implant holder.

In another embodiment of the invention, the propulsion device comprises a winding cylinder attached to the holder for pulling the carrier through the opening.

In another embodiment of the invention, the carrier extends through the open end of the holder and is attached to the winding cylinder.

In another embodiment of the invention, a crank is attached to the winding cylinder for rotating the winding cylinder and pulling the carrier through the end of the holder.

In accordance with a method embodiment of the invention, the method includes the steps of providing a holder for receiving an implant and having an open downstream end dimensioned to be inserted into the incision and for passing the implant therethrough. A flexible carrier is provided that extends through the downstream end of the implant holder and is positioned along inner and outer wall surfaces of the holder for frictionally-engaging the implant at least at a position on the inner wall surface proximate the downstream end of the holder. The carrier and the holder are configured such that when the carrier is caused to move through the open downstream end, the implant moves with the carrier, through the opening, and into the incision. The implant is placed in the flexible carrier and the flexible carrier and the implant are then placed into the holder. Then the downstream end of the holder is placed into the incision. The carrier is caused to move through the downstream end such that the implant is propelled from the holder into the incision.

In accordance with another method embodiment of the invention, the method includes the steps of providing a tubular implant holder for retaining an implant therein. The tubular implant holder has a downstream, open end dimensioned to be inserted into the incision and for the implant to be passed in a downstream direction through the open end from a position upstream therefrom. A flexible carrier is also provided that extends through the open end of the implant holder and is positioned along inner and outer wall surfaces of the implant holder. The flexible implant holder is configured to frictionally engage the implant at least at a position on the inner wall surface proximate the open end of the implant holder. A propulsion device cooperating with the flexible carrier adapted for propelling the implant from within the implant holder through the open end and into the incision. The implant is propelled by upstream movement of the flexible carrier relative to the outer wall surface of the implant holder and simultaneous corresponding downstream implant-engaging movement of the flexible carrier relative to the inner wall surface of the implant holder. The implant is placed in the flexible carrier. The flexible carrier and the implant are placed into the holder. The downstream, open end of the holder is placed into the incision, and the propulsion device is operated such that the carrier is pulled out of the downstream end of the holder and the implant is propelled from the holder into the incision.

In another method embodiment, the flexible carrier is attached to the propulsion device.

In another method embodiment, the propulsion device comprises a winding cylinder.

In another method embodiment, the carrier is doubled over itself along the walls of the holder when the propulsion device is operated.

In another method embodiment, the implant is inserted into the incision through the holder without the implant contacting skin.

### BRIEF DESCRIPTION OF THE DRAWINGS

Some of the objects of the invention have been set forth above. Other objects and advantages of the invention will appear as the description of the invention proceeds when taken in conjunction with the following drawings, in which:

FIG. 1 is a perspective view of an implant insertion device according to one embodiment of the invention;

FIG. 2 is another perspective view of an implant insertion device according to one embodiment of the invention;

FIG. 3 is a side view of the implant insertion device shown in FIGS. 1 and 2;

FIGS. 4 and 5 are views of the carrier within which the implant is positioned for use with the insertion device; and

FIG. 6 is a partial cross-sectional view of the implant insertion device with an implant positioned in a carrier within the insertion device.

### DESCRIPTION OF THE PREFERRED EMBODIMENTS AND BEST MODE

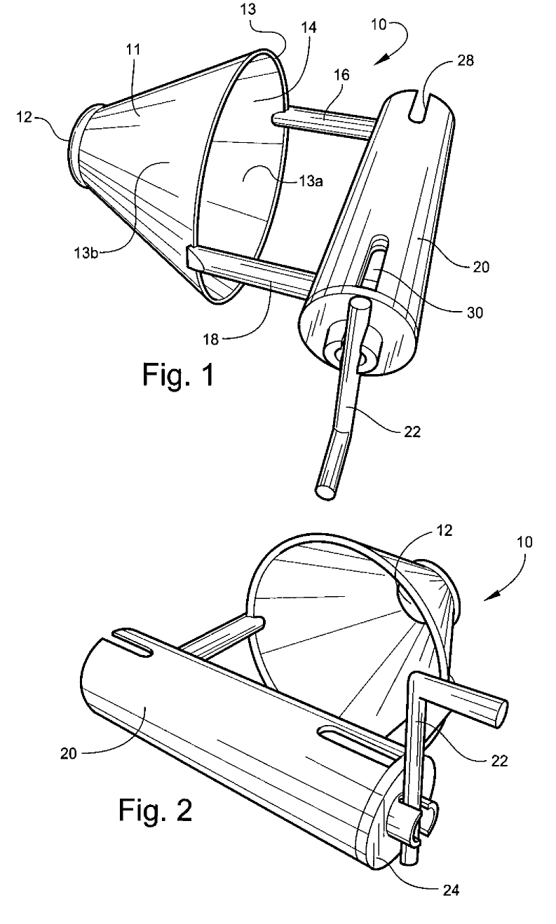

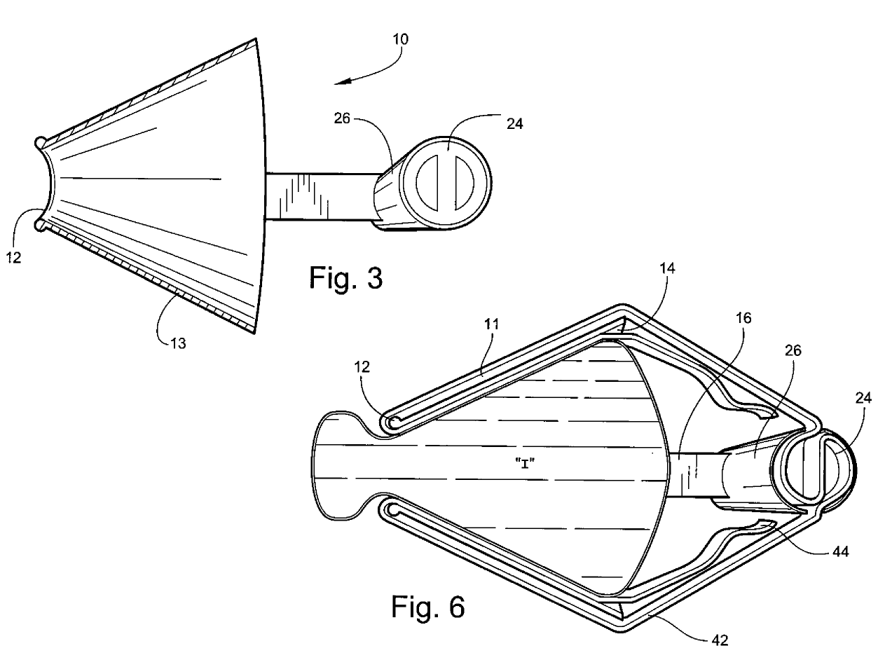

Referring now specifically to the drawings, an insertion device, without an implant carrier, according to an embodiment of the present invention is shown generally in FIGS. 1, 2, and 3 at reference numeral 10. Insertion device 10 includes a funnel 11 having a relatively small diameter distal end opening 12 that is located downstream of a relatively large diameter proximal end opening 14. The funnel 11 has a wall 13 that has an inner wall surface 13a and an outer wall surface 13b. The funnel 11 is attached by a pair of attachment braces 16, 18 to a propulsion device in the form of a hand crank 20. Hand crank 20 includes a handle 22 that is used to rotate a winding cylinder 24 contained within a cylindrical housing 26. Slots 28, 30 in the housing 26 allow access to the winding cylinder 24, as described in further detail below. The distal and proximal end openings 12, 14 may be circular or non-circular. A small motor or other manually-operated device may be used instead of the hand crank 20.

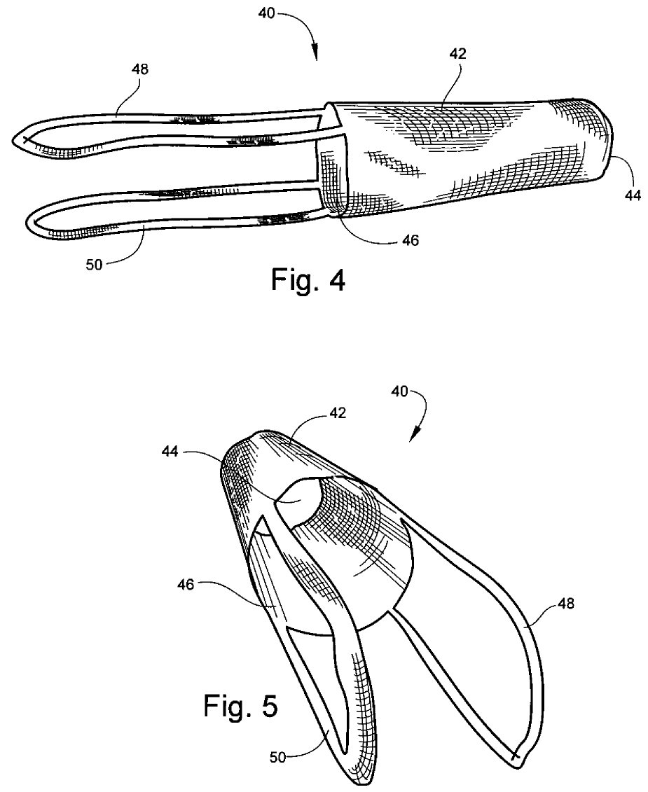

Referring now to FIGS. 4 and 5, an implant carrier 40 is shown, and comprises a flexible elongated sleeve 42 having a relatively small end opening 44 and a relatively large end opening 46. A pair of loops 48, 50 are formed with or attached to the sleeve 42 at the large end opening 46. The implant carrier 40 is preferably formed of a suitable surgical fabric, such as a synthetic knitted material, or other knitted, woven, non-woven or sheet material that can be sterilized, is flexible and is otherwise suitable for use in a surgical environment.

The carrier 40 is used in conjunction with the insertion device 10 to insert an implant a body through a suitably-sized incision. In one embodiment, the implant is inserted through the incision into a pocket formed in a breast. As is shown in FIG. 6, an implant, such as a breast implant “I” is first positioned in the implant carrier 40, with both ends 44, 46 open. The large end 46 can be closed so long as the carrier 40 is longer than the implant to allow the implant to move backward during insertion. The smaller end 44 can also be closed but must be designed to open upon insertion. The loops 48, 50 should have little or no elasticity.

The carrier 40 is inserted into the funnel 11 of the insertion device 10. The loops 48, 50 are pulled through the distal end of the insertion device 10. The loops 48, 50 are then brought back over the outside of the funnel 11 and attached to the winding cylinder 24 contained within the cylindrical housing 26 through the slots 28, 30. The distal end opening 12 of the funnel 11 is placed against the incision through which the implant “I” will be inserted into the pocket formed in the breast. By turning the hand crank 22, the loops 48, 50 are wound onto the winding cylinder 24, pulling the sleeve 42 out through the distal end opening 12 and doubling the sleeve 42 over itself along the wall surfaces 13a, 13b of the funnel 11. As this occurs, the implant “I” is ejected from the distal end 12 of the funnel and into the pocket through the incision in the breast.

It is believed that the funnel 11 is the most appropriate shape to reduce the cross sectional area of an implant for insertion into a small orifice. The fundamental concept behind the apparatus and method described above is that the implant “I” is being pulled though the opening 12 in the funnel 11 by the carrier 40, rather than pushing as others have attempted. When the implant “I” is pulled, it temporarily distorts to a more elongated shape which is conductive to insertion. When the implant is pushed it becomes flat and thus much more difficult to pass through the funnel.

The silicone shell on the surface of the implant “I” has a very high coefficient of friction on most materials such as stainless steel, which is used in most surgical instruments. There is a slightly lower coefficient of friction with the fabric carrier 40, allowing an implant “I” to distort and move in the sleeve 42 of the carrier 40 as needed during ejection from the insertion device 10 into the breast.

The carrier 40 has a low coefficient of friction with the funnel 11. However, there is enough friction between the implant “I” and carrier 40 to pull the implant “I” through the funnel 11 with minimal slip. In contrast, the traditional hand insertion process produces focused areas of pressure on the implant where the finger pushes the implant into the wound. This pressure is extremely variable. The pressure changes with each pass of the finger, each case, each implant size, each wound size, and each surgeon. The process described above allows the pressure to be reduced and evenly distributed throughout the implant. More importantly, the process is completely reproducible and allows for control of amount of force applied to the implant during insertion, in a reproducible fashion. The carrier 40 also allows the implant “I” to be inserted with no contact to skin, gloves, or instrument. This can dramatically reduce bacterial, material, or chemical contamination, thus decreasing the rate of capsular contracture and infection.

Finally, the process is easier and faster than the traditional hand held technique. It may also reduce liability for the surgeon and implant complaints, while reducing the risk for implant rupture and contamination.

A method and apparatus for inserting a flexible implant into a human or animal body is described above. Various details of the invention may be changed without departing from the scope of the invention. Furthermore, the foregoing description of the preferred embodiment of the invention and best mode for practicing the invention are provided for the purpose of illustration only and not for the purpose of limitation, the invention being defined by the claims.

I claim:

1. An insertion device for inserting an implant into a body through an incision, the device comprising:
(a) a holder for receiving an implant and having an open downstream end dimensioned to be inserted into the incision and for passing the implant therethrough;
(b) a flexible carrier extending through the downstream end of the implant holder and positioned along inner and outer walls of the holder for frictionally-engaging the implant at least at a position on the inner wall surface proximate the downstream end of the holder; and
(c) wherein the carrier and the holder are configured such that when the carrier is caused to move through the open downstream end, the implant moves with the carrier, through the opening, and into the incision.

2. An insertion device according to claim 1, and further comprising a propulsion device cooperating with the flexible carrier for propelling the implant from within the implant holder through the downstream end and into the incision by movement of the flexible carrier such that a portion of the flexible carrier moves outside of the holder away from the downstream end and, simultaneously, another portion of the flexible carrier engages the implant and moves inside the holder toward the downstream end, thereby causing the implant to move toward the downstream end.

3. An insertion device according to claim 2, wherein the propulsion device comprises a winding cylinder for pulling the carrier through the opening, the winding cylinder being attached to the holder.

4. An insertion device according to claim 3, wherein the carrier extends through the downstream end of the holder and is attached to the winding cylinder.

5. An insertion device according to claim 4, further comprising a crank attached to the winding cylinder for rotating the winding cylinder, thereby pulling the carrier through the end of the holder.

6. An insertion device according to claim 1, wherein the carrier comprises a flexible sleeve that is dimensioned to receive the implant therein.

7. An insertion device according to claim 1, wherein the holder is funnel shaped and has an open upstream end that is larger than the downstream end.

8. An insertion device for inserting an implant into a body through an incision, comprising:
(a) a tubular implant holder for retaining an implant therein, and having a downstream, open end dimensioned to be inserted into the incision and the implant passed in a downstream direction through the open end from a position upstream therefrom;
(b) a flexible carrier extending through the open end of the implant holder and positioned along inner and outer wall surfaces of the implant holder for frictionally-engaging the implant at least at a position on the inner wall surface proximate the open end of the implant holder; and
(c) a propulsion device cooperating with the flexible carrier adapted for propelling the implant from within the implant holder through the open end and into the incision by upstream movement of the flexible carrier relative to the outer wall surface of the implant holder and simultaneous corresponding downstream implant-engaging movement of the flexible carrier relative to the inner wall surface of the implant holder.

9. An insertion device according to claim 8, wherein the propulsion device comprises a winding cylinder attached to the holder for pulling the carrier through the opening.

10. An insertion device according to claim 9, wherein the carrier extends through the open end of the holder and is attached to the winding cylinder.

11. An insertion device according to claim 10, and further comprising a crank attached to the winding cylinder for rotating the winding cylinder and pulling the carrier through the end of the holder.

12. An insertion device according to claim 8, wherein the carrier comprises a flexible sleeve that is dimensioned to receive the implant therein.

13. An insertion device according to claim 8, wherein the holder is funnel shaped and has an upstream open end that is larger than the downstream open end.

14. A method for inserting an implant into a body through an incision, comprising the steps of:
(a) providing a holder for receiving an implant and having an open downstream end dimensioned to be inserted into the incision and for passing the implant therethrough; a flexible carrier extending through the downstream end of the implant holder and positioned along inner and outer wall surfaces of the holder for frictionally-engaging the implant at least at a position on the inner wall surface proximate the downstream end of the holder; and wherein the carrier and the holder are configured such that when the carrier is caused to move through the open downstream end, the implant moves with the carrier, through the opening, and into the incision;
(b) placing the implant in the flexible carrier;
(c) placing the flexible carrier and the implant into the holder;
(d) placing the downstream end of the holder into the incision; and
(e) causing the carrier to move through the downstream end such that the implant is propelled from the holder into the incision.

15. A method for inserting an implant into a body through an incision, comprising the steps of:
(a) Providing a tubular implant holder for retaining an implant therein, and having a downstream, open end dimensioned to be inserted into the incision and the implant passed in a downstream direction through the open end from a position upstream therefrom; a flexible carrier extending through the open end of the implant holder and positioned along inner and outer wall surfaces of the implant holder for frictionally-engaging the implant at least at a position on the inner wall surface proximate the open end of the implant holder; and a propulsion device cooperating with the flexible carrier adapted for propelling the implant from within the implant holder through the open end and into the incision by upstream movement of the flexible carrier relative to the outer wall surface of the implant holder and simultaneous corresponding downstream implant-engaging movement of the flexible carrier relative to the inner wall surface of the implant holder;
(b) placing the implant in the flexible carrier;
(c) placing the flexible carrier and the implant into the holder;
(d) placing the downstream, open end of the holder into the incision; and
(e) operating the propulsion device such that the carrier is pulled out of the downstream end of the holder and the implant is propelled from the holder into the incision.

16. A method for inserting an implant into a body according to claim 15, wherein step (c) of placing the flexible carrier and the implant into the holder includes the step of attaching the flexible carrier to the propulsion device.

17. A method for inserting an implant into a body according to claim 15, wherein the propulsion device comprises a winding cylinder.

18. A method for inserting an implant an open downstream end a body according to claim 15, wherein the step of operating the propulsion device includes the step of doubling the carrier over itself along the walls of the holder.

19. A method for inserting an implant into a body according to claim 15, wherein the step of operating the propulsion device includes the step of inserting the implant into the incision through the holder without the implant contacting skin.
